(19) United States
(12) Patent Application Publication
(10) Pub. No.: US 2022/0023481 A1
(43) Pub. Date: Jan. 27, 2022

(54) STERILIZATION FILTRATION SYSTEM (SFS) METHODS AND EQUIPMENT

(71) Applicants: Huy V. Huynh, Johns Creek, GA (US); Yun Joo Huynh, Johns Creek, GA (US)

(72) Inventors: Huy V. Huynh, Johns Creek, GA (US); Yun Joo Huynh, Johns Creek, GA (US)

(21) Appl. No.: 16/936,298

(22) Filed: Jul. 22, 2020

(51) Int. Cl. A61L 9/14; A61L 9/20; A61L 9/04; A61L 9/22; A61L 9/014

(52) U.S. Cl. CPC A61L 9/145; A61L 9/20; A61L 9/046; A61L 9/22; A61L 2209/22; A61L 2209/14; A61L 2209/12; A61L 2209/212; A61L 9/014

(57) ABSTRACT

A method for extracting and capturing airborne aerosol and particulate matters in the air that may be potentially contaminated with harmful bacteria and viruses using a vacuum extraction system; treating the contaminated air by scrubbing it with a biocide solution; filtering the scrubbed air using activated carbon and HEPA filters to remove any residual biocide; and optionally, treating the filtered air with UV-C, ozone, or plasma, before the sterilized air is return to the environment.

### FIELD OF THE INVENTION

The technology described herein relates generally to methods, systems, and devices for air filtration. More specifically, technology described herein relates to a method for extracting and capturing airborne aerosol and particulate matters in the air that may be potentially contaminated with harmful bacteria and viruses. Furthermore, this technology relates to methods, systems, and devices for using a vacuum extraction system, treating the contaminated air by scrubbing it with a biocide solution, filtering the scrubbed air using activated carbon and HEPA filters to remove any residual biocide, and optionally, treating the filtered air with UV-C, ozone, or plasma, before the sterilized air is return to the environment.

### BACKGROUND OF THE INVENTION

The Sterilization Filtration System (“SFS”) technology described herein is developed to create personal protection equipment for medical workers such as Dentists and Hygienists who work in the confined space of an operatory where treatment procedures often generate aerosol and particulate matters from patients that may potentially carry transmittable diseases and virus. The SFS reduces or eliminates the aerosol and particulate matters using a vacuum extraction system that is connected to an articulated arm or a flexible hose with a collection funnel at the end. The air is passed through a biocide solution where the aerosol and particulate matters are entrained, and the potentially harmful bacteria and viruses are render harmless by the biocide. The scrubbed air is routed through HEPA and activated carbon filters before the sterilized air is returned to the room.

The SFS technology described herein is also developed to treat public or private spaces with large volume of air that is recirculated, and where airborne transmittable diseases may be present. Examples include airplane cabins, trains, buses, hospitals, commercial offices, retail stores, classrooms, churches, restaurants, public buildings, et cetera, where “The smallest particles (1- to 5-μm droplets nuclei) can remain much longer than cough droplets airborne for many minutes or even hours,” IES Photobiology Committee, “IES Committee Report: Germicidal Ultraviolet (GUV)—Frequently Asked Questions,” IES CR-2-20-V1, Apr. 15, 2020. For the larger areas, the air is drawn into a gas-liquid scrubber containing a biocide solution, where the aerosol and particulate matters are captured and entrained, and the potentially harmful bacteria and viruses are rendered harmless by the biocide. The scrubbed air is routed through HEPA and activated carbon filters before the sterilized air is returned to the room. An optional secondary treatment step wherein the filtered air is exposed to ultra-violet light, ozone, or ionized gas plasma may be desirable due to the large volume of air being treated. It should be noted that the gas-liquid scrubber principal is well understood to those who are familiar with the art and is not covered here, Perry, R. H., Green, D. W., Kawatra, S. K.; Eisele, T. C. Separation of Flue-Gas Scrubber Sludge into . . . Penney, W. R.; Crocker, B. B. “Gas Absorption and Gas-Liquid System Design,” Section 14, Perry's Chemical Engineers' Handbook, 7th Ed. Similarly, UV-C, ozone and ionized gas plasma technologies are widely documented

There are various devices, systems, and methods that are utilized for filtration. However, there are numerous deficiencies and shortcomings in the known mounting devices, systems, and methods that are utilized for air filtration for extracting and capturing airborne aerosol and particulate matters in the air that may be potentially contaminated with harmful bacteria and viruses.

A multi-stage air filtration system is widely available on the market, consisting of a circulation fan to draw the air through a series of chambers: HEPA filter to remove particulates, activated carbon to remove odor, UV radiation or ozone to kill germs, and air ionizer to electrostatically attract airborne charged particles. This method is effective for general use, but insufficient protection for infectious diseases and virus, “Studies at the Harvard School of Public Health^{13,14} and elsewhere show log units of reduction equivalent to 24 ACH to achieve 80% reduction of transmission. Of course, 100% reduction is not possible, because of the multiple modes of transmission,”,” IES Photobiology Committee, “IES Committee Report: Germicidal Ultraviolet (GUV)—Frequently Asked Questions,” IES CR-2-20-V1, Apr. 15, 2020.

Related utility patents known in the art include the following:

U.S. Pat. No. 10,632,217, issued to Axelson on Apr. 28, 2020, discloses a rapid surface disinfection method and apparatus.

U.S. Pat. No. 10,632,419, issued to Nakazawa et al. on Apr. 28, 2020, discloses a vacuum pump with abatement function.

U.S. Pat. No. 10,631,624, issued to Lenkiewicz et al. on Apr. 28, 2020, discloses a carpet and upholstery cleaning product.

U.S. Pat. No. 10,646,611, issued to Temple on May 12, 2020, discloses methods and equipment for treating industrial gas streams and biological fouling.

The foregoing patent and other information reflect the state of the art of which the inventor is aware and are tendered with a view toward discharging the inventor's acknowledged duty of candor in disclosing information that may be pertinent to the patentability of the technology described herein. It is respectfully stipulated, however, that the foregoing patent and other information do not teach or render obvious, singly or when considered in combination, the inventor's claimed invention.

### BRIEF SUMMARY OF THE INVENTION

This technology relates to methods, systems, and devices for using a vacuum extraction system, treating the contaminated air by scrubbing it with a biocide solution, filtering the scrubbed air using activated carbon and HEPA filters to remove any residual biocide, and optionally, treating the filtered air with UV-C, ozone, or plasma, before the sterilized air is return to the environment.

In at least one exemplary embodiment, the technology described herein includes a method to extract and sterilize air before return to the environment. The method includes: utilizing a vacuum system to extract and capture airborne aerosol and particulate matters in the air that may be potentially contaminated with harmful bacteria and viruses through an intake port; treating the contaminated air by passing it through a scrubbing solution containing a biocide that is capable of neutralizing and sterilizing the harmful bacteria and viruses; and filtering the scrubbed air using activated carbon and HEPA filters to remove any residual biocide.

In at least one embodiment, the method also includes treating the filtered air with UV-C.

In at least one embodiment, the method also includes treating the filtered air with ozone.

In at least one embodiment, the method also includes treating the filtered air with plasma.

In at least one embodiment, the method also includes connecting the vacuum system to an articulated arm with a collection funnel, a flexible hose with a collection funnel, or a static intake vent.

In at least one embodiment, the method also includes scrubbing the contaminated aerosols and particulate matters by bubbling the air through a biocide solution, or by passing the air through a bed of packing material that is wetted with a biocide solution.

In at least one embodiment, the method also includes utilizing a biocide scrubbing solution containing one or more chemical substance or microorganism that are intended to destroy, deter, render harmless, or exert a controlling effect on any harmful organism as defined by Regulations (EU Biocidal Products Regulation (BPR), EU No 528/2012, and the US Federal Insecticide, Fungicide and Rodenticide Act (FIFRA), 40 CFR Part 147).

In at least one embodiment, the method also includes passing the scrubbed air through a filter before the sanitized air to return to the environment.

In at least one embodiment, the method also includes utilizing the vacuum system to pull or push the air through a scrubber solution and a filter media.

In at least one embodiment, the method also includes utilizing a filter comprised of one or more combination of a High Efficiency Particulate Air (HEPA) filter, an activated carbon filter, and a filter media coated with anti-microbial substance.

In at least one embodiment, the method also includes utilizing a secondary treatment step by exposing the filter air to germicidal ultraviolet light (UV-C, 200-280 nanometers), ozone, or ionized gas plasma before the sanitized air to return to the environment.

In another exemplary embodiment, the technology described herein includes system to extract and sterilize air before return to the environment. The system includes: a vacuum system to extract and capture airborne aerosol and particulate matters in the air that may be potentially contaminated with harmful bacteria and viruses through an intake port; a scrubbing solution containing a biocide to treat the contaminated air as it is passed through to neutralize and sterilize any harmful bacteria and viruses; and a filter to scrub air with activated carbon and HEPA filters to remove any residual biocide.

In at least one embodiment, the system also includes a source of UV-C to treat the filtered air.

In at least one embodiment, the system also includes a source of ozone to treat the filtered air.

In at least one embodiment, the system also includes a source of plasma to treat the filtered air.

In at least one embodiment, the system also includes an articulated arm with a collection funnel, a flexible hose with a collection funnel, or a static intake vent to connect to the vacuum system.

In at least one embodiment, the system also includes a bed of packing material that is wetted with the biocide solution to treat the contaminated air as it is passed through to neutralize and sterilize any harmful bacteria and viruses.

In at least one embodiment, the system also includes a biocide scrubbing solution comprising one or more chemical substance or microorganism that are intended to destroy, deter, render harmless, or exert a controlling effect on any harmful organism as defined by Regulations (EU Biocidal Products Regulation (BPR), EU No 528/2012, and the US Federal Insecticide, Fungicide and Rodenticide Act (FIFRA), 40 CFR Part 147).

In at least one embodiment, the system also includes wherein the filter comprises one or more combination of a High Efficiency Particulate Air (HEPA) filter, an activated carbon filter, and a filter media coated with anti-microbial substance.

There has thus been outlined, rather broadly, the more important features of the technology in order that the detailed description thereof that follows may be better understood, and in order that the present contribution to the art may be better appreciated. There are additional features of the technology that will be described hereinafter and which will form the subject matter of the claims appended hereto. In this respect, before explaining at least one embodiment of the technology in detail, it is to be understood that the invention is not limited in its application to the details of construction and to the arrangements of the components set forth in the following description or illustrated in the drawings. The technology described herein is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the technology described herein. It is important, therefore, that the claims be regarded as including such equivalent constructions insofar as they do not depart from the spirit and scope of the technology described herein.

Further objects and advantages of the technology described herein will be apparent from the following detailed description of a presently preferred embodiment which is illustrated schematically in the accompanying drawings.

### BRIEF DESCRIPTION OF THE DRAWINGS

The technology described herein is illustrated with reference to the various drawings, in which like reference numbers denote like device components and/or method steps, respectively, and in which:

FIG. 1A is a schematic view of a vacuum system for use in the sterilization filtration system, illustrating, in particular, key components, according to an embodiment of the technology described herein;

FIG. 1B is a schematic view of a biocide scrubber for use in the sterilization filtration system, illustrating, in particular, key components, according to an embodiment of the technology described herein;

FIG. 1C is a schematic view of an extraction arm for use in the sterilization filtration system, illustrating, in particular, key components, according to an embodiment of the technology described herein; and

FIG. 2 is a schematic view of a large biocide scrubber for use in the sterilization filtration system, illustrating, in particular, key components, according to an embodiment of the technology described herein.

### DETAILED DESCRIPTION OF THE INVENTION

Before describing the disclosed embodiments of this technology in detail, it is to be understood that the technology is not limited in its application to the details of the particular arrangement shown here since the technology described is capable of other embodiments. Also, the terminology used herein is for the purpose of description and not of limitation.

In the following description, specific details are set forth in order to provide a thorough understanding of this SFS invention. On the one hand, it is understood that those who are skilled in the art may practice this SFS invention without these specific details. On the other hand, well-known methods, procedures, and components have not been described in detail so as not to obscure this SFS invention.

The SFS invention consists of three primary modules, and a fourth optional add-on process for large system: 1) Vacuum system, 2) Biocide Scrubber 3) Filter, and 4) UV-C, ozone, or ionized gas plasma.

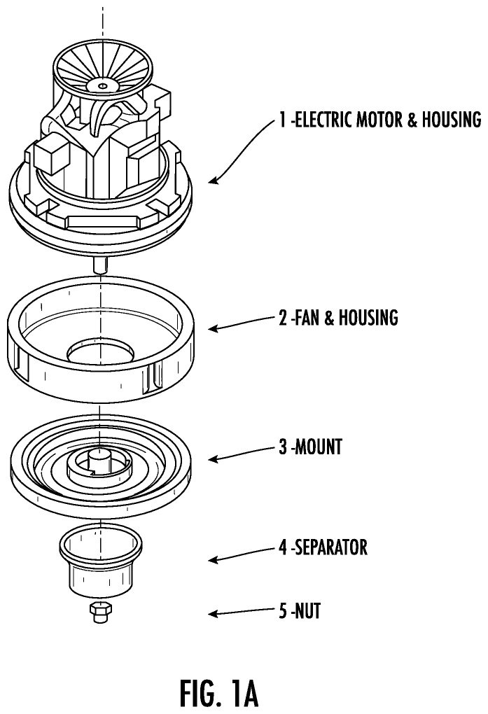

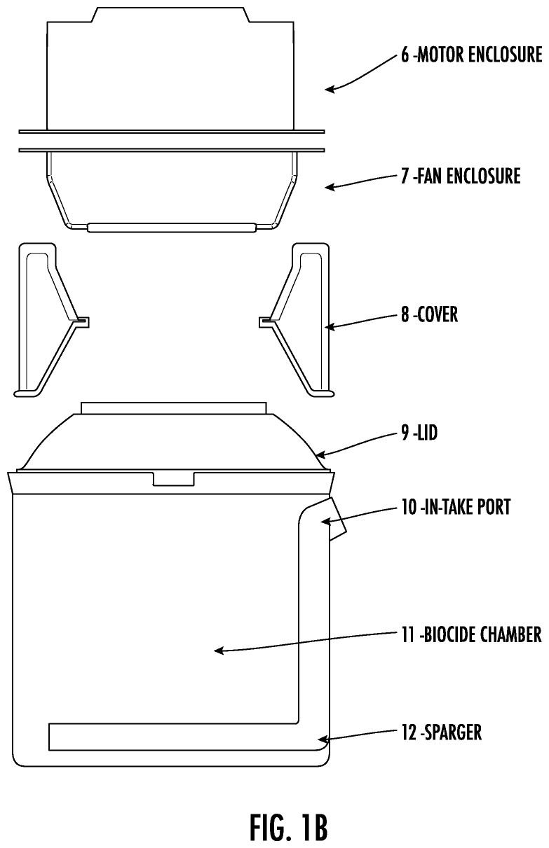

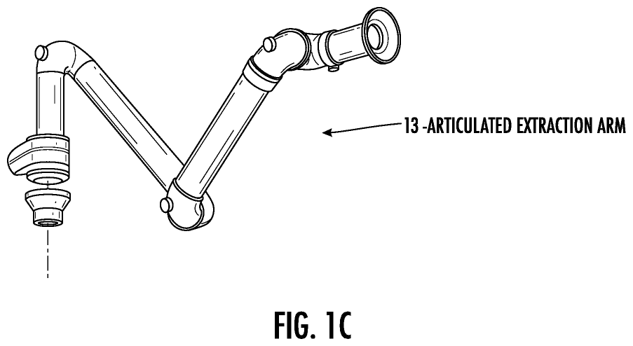

A portable, compact SFS unit may be employed for point source generators such as dental operatories. A portable SFS unit consists of a vacuum system to draw in air and capture the contaminated airborne aerosol and particulate matters from propagating through the air, FIG. 1(a). An articulated arm may be used to position the vacuum port as close to the emission source as possible, FIG. 1(c). The contaminated air is drawn through a biocide solution, FIG. 1(c). A sparger is employed to ensure thorough optimal gas and liquid contact, FIG. 1(b). A dynamic filtration technology is used to separate the gas from the liquid before the scrubbed air is routed through HEPA and activated carbon filters to remove the residual biocide in the sterilized air.

In at least one embodiment, the vacuum system depicted in FIG. 1A includes an electric motor and housing 1, a fan and housing 2, a mount 3, a separator 4, and a nut 5.

In at least one embodiment, the biocide scrubber depicted in FIG. 1B includes a motor closure 6, a fan enclosure 7, a cover 8, a lid 9, an in-take port 10, a biocide chamber 11, and a sparger 12.

In at least one embodiment, the extraction arm depicted in FIG. 1C includes an articulated extraction arm 13.

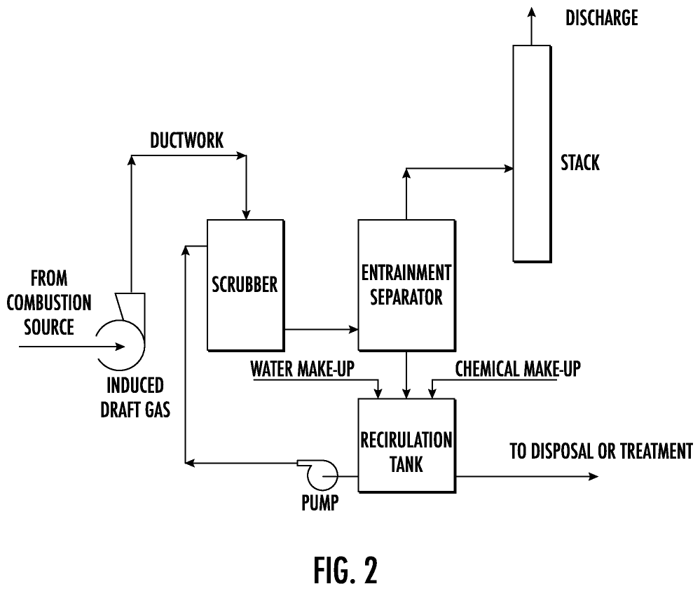

The SFS can be used to sterilize airborne pathogens in larger public spaces where air is recycled, e.g., airplane cabins, trains, buses, hospitals, commercial offices, retail stores, classrooms, churches, restaurants, public buildings, et cetera. A vacuum system is required to pull the contaminated air through the scrubber solution. Alternatively, the contaminated air may also be pushed through the scrubber solution. The rate of air flow depends on the volume of air requiring sterilization. Aerating air through a liquid solution has practical limitation with equipment size and weight for an aeration and gas-liquid separation system at high gas flow due to a phenomenon known as 2-phase flow to those who are acquainted with the art. Therefore, a classical gas-liquid wet scrubber design is employed in lieu of aeration, FIG. 2. The wet scrubber principle and designs are well understood by those who are skilled in the art, hence it is not addressed further in this patent. Once the contaminated air is treated by the biocide solution, the scrubbed air is drawn through HEPA and activated filters to remove any residual biocides that may be present in the sanitized air stream. Depending on the system requirements such as volumetric air flow rate, the biocide solution, and scrubber design, a supplemental treatment step using UV-C radiation, ozone, or plasma may be added to ensure that the air is adequately sterilized. UV-C radiation, ozone, and plasma are well understood by those who are skilled in the art, hence it is not addressed further in this patent.

Although this technology has been illustrated and described herein with reference to preferred embodiments and specific examples thereof, it will be readily apparent to those of ordinary skill in the art that other embodiments and examples can perform similar functions and/or achieve like results. All such equivalent embodiments and examples are within the spirit and scope of the technology described herein and are intended to be covered by the following claims.

What is claimed is:

1. A method to extract and sterilize air before return to the environment, the method comprising:
utilizing a vacuum system to extract and capture airborne aerosol and particulate matters in the air that may be potentially contaminated with harmful bacteria and viruses through an intake port;
treating the contaminated air by passing it through a scrubbing solution containing a biocide that is capable of neutralizing and sterilizing the harmful bacteria and viruses; and
filtering the scrubbed air using activated carbon and HEPA filters to remove any residual biocide.

2. The method of claim 1, further comprising:
treating the filtered air with UV-C.

3. The method of claim 1, further comprising:
treating the filtered air with ozone.

4. The method of claim 1, further comprising:
treating the filtered air with plasma.

5. The method of claim 1, further comprising:
connecting the vacuum system to an articulated arm with a collection funnel, a flexible hose with a collection funnel, or a static intake vent.

6. The method of claim 1, further comprising:
scrubbing the contaminated aerosols and particulate matters by bubbling the air through a biocide solution, or by passing the air through a bed of packing material that is wetted with a biocide solution.

7. The method of claim 1, further comprising:
utilizing a biocide scrubbing solution containing one or more chemical substance or microorganism that are intended to destroy, deter, render harmless, or exert a controlling effect on any harmful organism as defined by Regulations (EU Biocidal Products Regulation (BPR), EU No 528/2012, and the US Federal Insecticide, Fungicide and Rodenticide Act (FIFRA), 40 CFR Part 147).

8. The method of claim 1, further comprising:
passing the scrubbed air through a filter before the sanitized air to return to the environment.

9. The method of claim 5, further comprising:
utilizing the vacuum system to pull or push the air through a scrubber solution and a filter media.

10. The method of claim 8, further comprising:
utilizing a filter comprised of one or more combination of a High Efficiency Particulate Air (HEPA) filter, an activated carbon filter, and a filter media coated with anti-microbial substance.

11. The method of claim 8, further comprising:
utilizing a secondary treatment step by exposing the filter air to germicidal ultraviolet light (UV-C, 200-280 nanometers), ozone, or ionized gas plasma before the sanitized air to return to the environment.

12. A system to extract and sterilize air before return to the environment, the system comprising:
vacuum system to extract and capture airborne aerosol and particulate matters in the air that may be potentially contaminated with harmful bacteria and viruses through an intake port;
a scrubbing solution containing a biocide to treat the contaminated air as it is passed through to neutralize and sterilize any harmful bacteria and viruses; and
a filter to scrub air with activated carbon and HEPA filters to remove any residual biocide.

13. The system of claim 12, further comprising:
a source of UV-C to treat the filtered air.

14. The system of claim 12, further comprising:
a source of ozone to treat the filtered air.

15. The system of claim 12, further comprising:
a source of plasma to treat the filtered air.

16. The system of claim 12, further comprising:
an articulated arm with a collection funnel, a flexible hose with a collection funnel, or a static intake vent to connect to the vacuum system.

17. The system of claim 12, further comprising:
a bed of packing material that is wetted with the biocide solution to treat the contaminated air as it is passed through to neutralize and sterilize any harmful bacteria and viruses.

18. The system of claim 12, further comprising:
a biocide scrubbing solution comprising one or more chemical substance or microorganism that are intended to destroy, deter, render harmless, or exert a controlling effect on any harmful organism as defined by Regulations (EU Biocidal Products Regulation (BPR), EU No 528/2012, and the US Federal Insecticide, Fungicide and Rodenticide Act (FIFRA), 40 CFR Part 147).

19. The system of claim 12, wherein the filter comprises one or more combination of a High Efficiency Particulate Air (HEPA) filter, an activated carbon filter, and a filter media coated with anti-microbial substance.
